(19) United States
(12) Patent Application Publication
(10) Pub. No.: US 2017/0087296 A1
(43) Pub. Date: Mar. 30, 2017

(54) MULTIPLE INTRAVENOUS LINE RETENTION SYSTEM

(71) Applicant: Rolanda Florence, Los Angeles, CA (US)

(72) Inventor: Rolanda Florence, Los Angeles, CA (US)

(21) Appl. No.: 14/871,435

(22) Filed: Sep. 30, 2015

(51) Int. Cl. A61M 5/158; A61M 25/02

(52) U.S. Cl. CPC A61M 5/158; A61M 25/02; A61M 2025/0206; A61M 2005/1586

(57) ABSTRACT

A multiple intravenous line retention system includes a flexible panel having a top side, a bottom side, a first end and a second end. The panel has a length from the first end to the second end that is greater than 3.0 inches and less than 11.0 inches. The panel has a width from a first lateral edge to a second lateral edge is less than 1.0 inches. The panel is comprised of a flexible material. A mount is attached to the top side of the panel and extends upwardly therefrom. The mount removably receives a plurality of intravenous lines. A coupler is attached to the panel and couples the panel to a patient receiving the intravenous lines.

### BACKGROUND OF THE DISCLOSURE

### Field of the Disclosure

The disclosure relates to intravenous line securing devices and more particularly pertains to a new intravenous line securing device for retaining intravenous lines in a secure condition.

### SUMMARY OF THE DISCLOSURE

An embodiment of the disclosure meets the needs presented above by generally comprising a panel having a top side, a bottom side, a first end and a second end. The panel has a length from the first end to the second end that is greater than 3.0 inches and less than 11.0 inches. The panel has a width from a first lateral edge to a second lateral edge is less than 1.0 inches. The panel is comprised of a flexible material. A mount is attached to the top side of the panel and extends upwardly therefrom. The mount removably receives a plurality of intravenous lines. A coupler is attached to the panel and couples the panel to a patient receiving the intravenous lines.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front perspective view of a multiple intravenous line retention system according to an embodiment of the disclosure.

FIG. 2 is a rear view of an embodiment of the disclosure.

FIG. 3 is a rear view of an embodiment of the disclosure.

FIG. 4 is a bottom view of an embodiment of the disclosure.

FIG. 5 is a top in-use view of the embodiment of FIG. 1 of the disclosure.

FIG. 6 is a front perspective view of an embodiment of the disclosure.

### DESCRIPTION OF THE PREFERRED EMBODIMENT

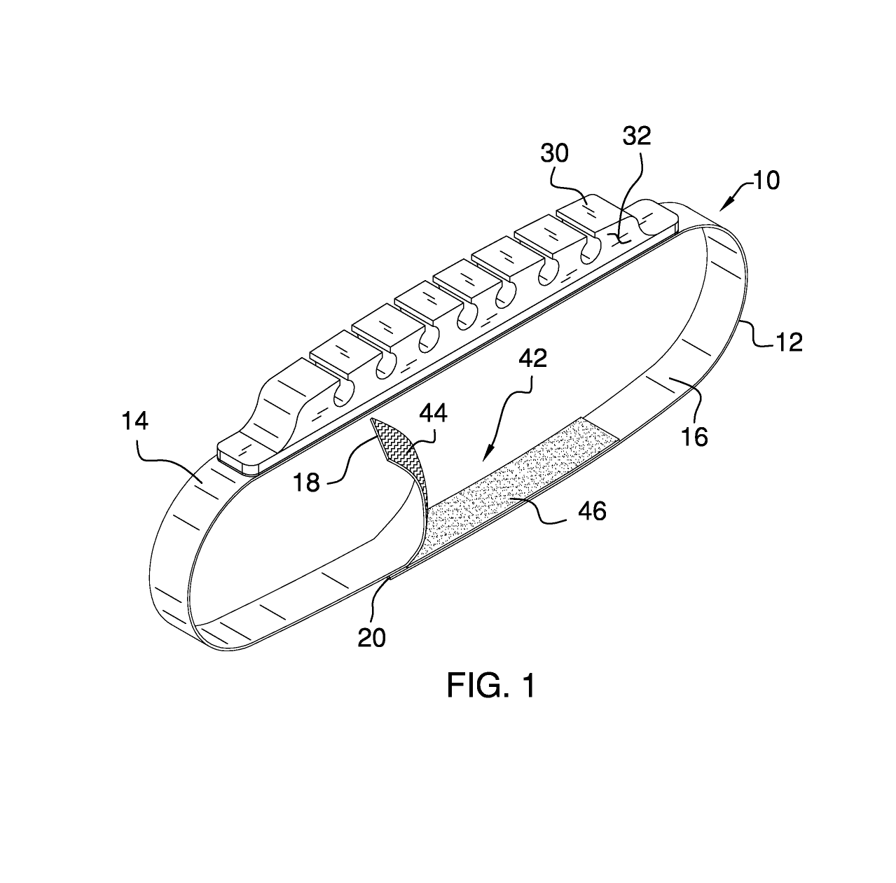

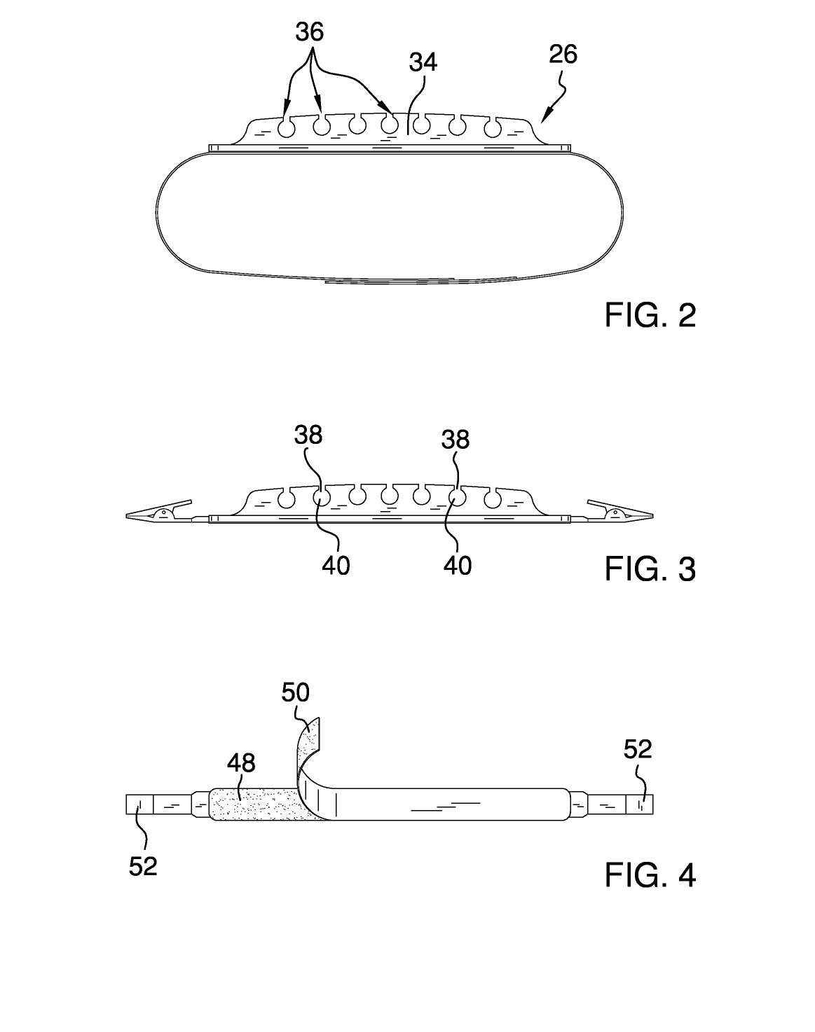

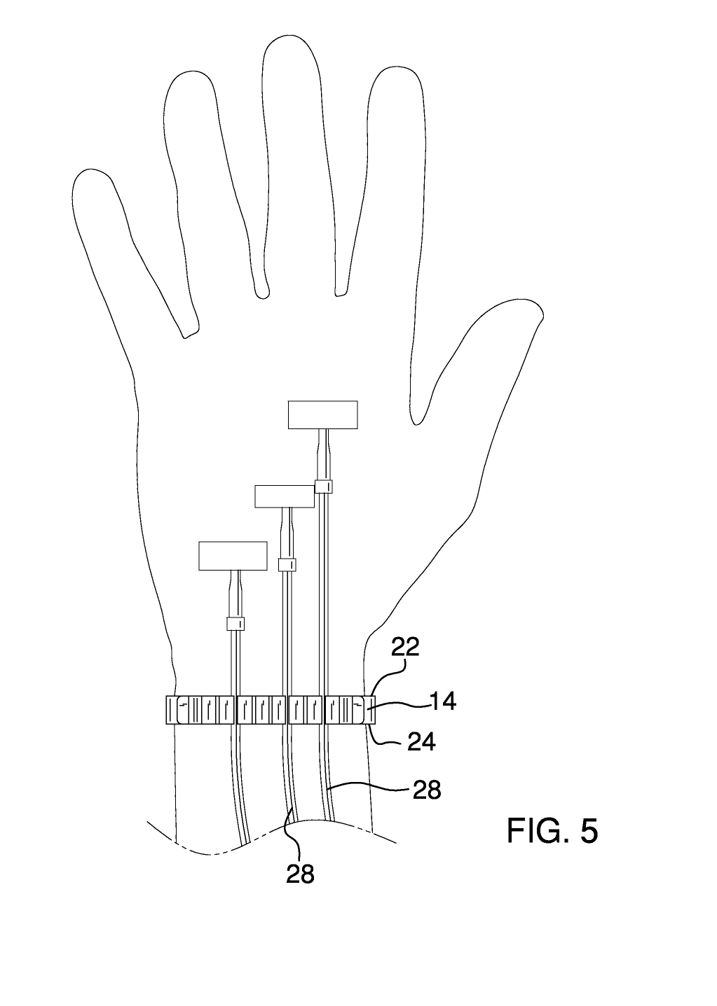

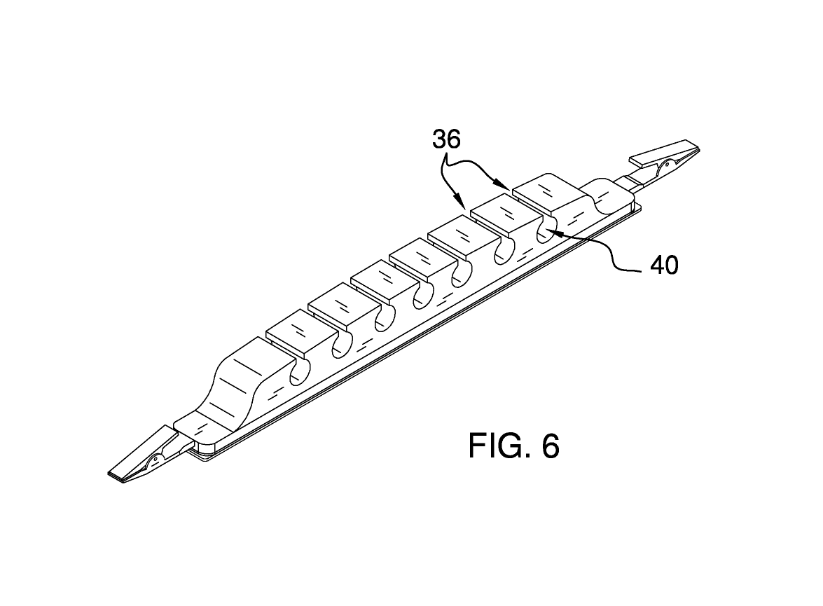

With reference now to the drawings, and in particular to FIGS. 1 through 6 thereof, a new intravenous line securing device embodying the principles and concepts of an embodiment of the disclosure and generally designated by the reference numeral 10 will be described.

As best illustrated in FIGS. 1 through 6, the multiple intravenous line retention system 10 generally comprises a panel 12 that has a top side 14, a bottom side 16, a first end 18 and a second end 20. The panel 12 has a length from the first end 18 to the second end 20 that is greater than 3.0 inches and less than 11.0 inches. The panel 12 has a width from a first lateral edge 22 to a second lateral edge 24 that may be less than 1.0 inches. The panel 12 is comprised of a flexible material which may be, for instance, a solid plastic material, woven nylon, or the like.

A mount 26 is attached to the top side 14 of the panel 12 and extends upwardly therefrom. The mount 26 is configured to removably receive a plurality of intravenous lines 28; that is tubes used for delivering a fluid intravenously. The mount 26 has an upper surface 30, a front surface 32 and a rear surface 34. The upper surface 30 has a plurality of breaks 36 therein extending downwardly toward the panel 12 and through each of the front 32 and rear 34 surfaces. Each break 36 has an upper portion 38 adjacent to the upper surface 30 and a lower portion 40 positioned between the upper portion 38 and the panel 12. Each lower portion 40 is larger than an associated one of the upper portions 38. The lower portions 40 each have a cylindrical shape. The mount 26 is comprised of a resiliently bendable material.

A coupler 42 is attached to the panel 12 and is configured to couple the panel 12 to a patient receiving the intravenous lines 28. The coupler 42 may comprise a hook and loop coupler releasably coupling the panel 12 together in a closed loop wherein a first mating member 44 is attached to the top side 14 adjacent to the first end 18 and a second mating member 46 is attached to the bottom side 16 adjacent to the second end 20 such that the top 14 and bottom 16 sides are overlapped to engage each other. The closed loop would typically be extended around the wrist of a patient as shown in FIG. 5. Alternatively, the coupler 42 may comprise an adhesive 48 attached to the bottom side 16 of the panel 12. The adhesive 48 may be covered with a removable sheet 50 to protect the adhesive 48 until use. The adhesive 48 may be used to attach the panel 12 to the skin of the patient or to an object or clothing of the patient. Yet another coupler 42 may include a pair of clips 52 wherein each of the first 14 and second 16 ends has one of the clips 52 attached thereto. The clips 52 are conventional spring clips and may be used to secure the panel 12 to an article of clothing adjacent to the wearer's wrist.

In use, panel 12 is attached to the patient in one of the manner described above so that the panel 12 is relatively near the place where there intravenous lines 28 enter the patient. The lines 28 are then placed in the mount 26 so that they are not easily movable relative to their insertion point and so that they do not become entangled with each other or hurt the patient by their movement. This prevents the need for taping each intravenous line 28 to a person as is typically done.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An intravenous line securing assembly configured to secure a plurality of intravenous lines being received by a patient, said assembly comprising:
a panel having a top side, a bottom side, a first end and a second end, said panel having a length from said first end to said second end being greater than 3.0 inches and less than 11.0 inches, said panel having a width from a first lateral edge to a second lateral edge being less than 1.0 inches, said panel being comprised of a flexible material;
a mount being attached to said top side of said panel and extending upwardly therefrom, said mount being configured to removably receive a plurality of intravenous lines; and
a coupler being attached to said panel and being configured to couple said panel to a patient receiving the intravenous lines.

2. The intravenous line securing assembly according to claim 1, wherein said mount has an upper surface, a front surface and a rear surface, said upper surface having a plurality of breaks therein extending downwardly toward said panel and through each of said front and rear surfaces.

3. The intravenous line securing assembly according to claim 2, wherein each break has an upper portion adjacent to said upper surface and a lower portion positioned between said upper portion and said panel, each lower portion being larger than an associated one of said upper portions.

4. The intravenous line securing assembly according to claim 3, wherein said lower portions each have a cylindrical shape.

5. The intravenous line securing assembly according to claim 4, wherein said mount is comprised of a resiliently bendable material.

6. The intravenous line securing assembly according to claim 2, wherein said mount is comprised of a resiliently bendable material.

7. The intravenous line securing assembly according to claim 1, wherein said coupler comprises a hook and loop coupler releasably coupling said panel together in a closed loop.

8. The intravenous line securing assembly according to claim 1, wherein said coupler comprises an adhesive being attached to said bottom side of said panel.

9. The intravenous line securing assembly according to claim 1, wherein said coupler comprises a pair of clips, each of said first and second ends having one of said clips attached thereto.

10. An intravenous line securing assembly configured to secure a plurality of intravenous lines being received by a patient, said assembly comprising:
a panel having a top side, a bottom side, a first end and a second end, said panel having a length from said first end to said second end being greater than 3.0 inches and less than 11.0 inches, said panel having a width from a first lateral edge to a second lateral edge being less than 1.0 inches, said panel being comprised of a flexible material;
a mount being attached to said top side of said panel and extending upwardly therefrom, said mount being configured to removably receive a plurality of intravenous lines, said mount having an upper surface, a front surface and a rear surface, said upper surface having a plurality of breaks therein extending downwardly toward said panel and through each of said front and rear surfaces, each break having an upper portion adjacent to said upper surface and a lower portion positioned between said upper portion and said panel, each lower portion being larger than an associated one of said upper portions, said lower portions each having a cylindrical shape, said mount being comprised of a resiliently bendable material; and
a coupler being attached to said panel and being configured to couple said panel to a patient receiving the intravenous lines, said coupler comprising a hook and loop coupler releasably coupling said panel together in a closed loop.
